(19) United States
(12) Patent Application Publication
(10) Pub. No.: US 2022/0137077 A1
(43) Pub. Date: May 5, 2022

(54) Flow Cytometry Measurement Method and Kit for Carrying Out Same

(71) Applicant: AVA Lifescience GmbH, Denzlingen (DE)

(72) Inventors: Holger Klapproth, Freiburg im Breisgau (DE); Ulrich Birsner, Oberried (DE); Marc Kessemeier, Emmendingen (DE)

(21) Appl. No.: 17/435,160

(22) PCT Filed: Mar. 1, 2019
(86) PCT No.: PCT/EP2019/055142

§ 371 Date: Aug. 31, 2021

(51) Int. Cl. G01N 33/96; G01N 33/543; G01N 33/544; G01N 33/58; G01N 33/50; G01N 15/14

(52) U.S. Cl. CPC G01N 33/96; G01N 33/54313; G01N 33/54353; G01N 2015/0065; G01N 33/582; G01N 33/5094; G01N 15/1434; G01N 33/544

(57) ABSTRACT

In a flow cytometry measurement method, an analysis medium is provided, which includes a fluid and biological cells contained therein. A labeling molecule is provided and is brought in contact with the analysis medium in such a way that the labeling molecule can bind specifically to a target structure located on the surface of the cell if the cell has said cell structure. For the individual cells, flow cytometry measured values are captured for a first and a second physical parameter. The first parameter is fluorescence radiation emitted by the labeling molecule when the labeling molecule is excited. The cells are classified on the basis of the flow cytometry measured values. A first calibrator and a second calibrator are provided, which have solid particles matching in shape, size and material. A target structure matching the target structure of the cells is immobilized on the surface of the first calibrator. The second calibrator does not have said target structure. The calibrators are mixed with the analysis medium before the flow cytometry measured values are captured. Corresponding first and second flow cytometry measured values are captured for the calibrators as well as for the cells. A normalized first flow cytometry measured value for the cell is formed from the first flow cytometry measured value of the first calibrator, the first flow cytometry measured value of the second calibrator and the first flow cytometry measured value of the cell.

### CROSS-REFERENCE TO RELATED APPLICATION

This application is the U.S. national phase of International Application No. PCT/EP2019/055142 filed Mar. 1, 2019, the disclosure of which is hereby incorporated by reference in its entirety.

### BACKGROUND OF THE INVENTION

### Field of the Invention

The invention relates to a flow cytometry measuring method in which an analysis medium is provided which has a fluid and biological cells to be classified contained therein, wherein at least one labeling molecule is provided and brought into contact with the analysis medium in such a way that the labeling molecule targets can specifically bind to at least one target structure located on the surface of the cells if the cell has this target structure, wherein a fluid flow of the analysis medium is generated in which the cells individually come into a measurement region of an energy beam and/or an electric field, wherein at least one first and one second flow cytometry measured value are captured for a first and second physical parameter, respectively, for the individual cells located in the measurement region, wherein at least the first parameter is a fluorescent radiation which the at least one labeling molecule emits when excited with the energy beam or the electric field, and wherein the cells are classified on the basis of the flow cytometry measured values. The invention also relates to a kit for carrying out the method.

### Description of Related Art

The term flow cytometry (cytometry: cell measurement) describes a measurement method that is used in biology and medicine. It allows the analysis of biological cells that pass through an electric field or a light beam one at a time at high speed. Depending on the shape, structure and/or color of the cells, different flow cytometry measured values are captured, from which the properties of the individual cells can be derived and the cells can thus be classified.

In one form of flow cytometry, fluorescence-labeled cells are sorted into different reagent vessels depending on the color thereof. Corresponding devices are called flow sorters or FACS (fluorescence-activated cell sorting). Sometimes the term FACS (=fluorescence-activated cell scanning) is used with this meaning for devices that do not sort the cells, but only perform an analysis or classification of the properties thereof.

The principle of the investigation is based on the emission of optical signals by the cell when it passes a laser beam. Focused by an enveloping current, the sample enters the microchannel of a high-precision cuvette made of glass or quartz, in such a way that each cell is guided one after the other through the measurement region of a laser beam. The resulting scattered light or fluorescence signal is captured by a detector. The result is quantitative information about each individual cell. By analyzing a large number of cells within a very short time interval (>1000 cells/second), representative information about cell populations contained in the analysis medium is quickly obtained.

At the same time as the scattered light, fluorescent colors can be measured in the flow cytometer. Only a few cells emit fluorescent light per se. Therefore, one uses labeling molecules, such as dyes, which bind to certain target structures of the cells. If, for example, the dyes DAPI (4′,6-diamidine-2′-phenylindole dihydrochloride) and propidium iodide are used, which bind to the deoxyribonucleic acid (DNA) of a cell (DAPI) or intercalate thereinto (propidium iodide)—i.e., be inserted between the bases—one can use the brightness of the fluorescent radiation that the at least one labeling molecule, which is immobilized on the cell, transmits when passing through the laser beam, to determine how much DNA the cell contains.

Antibodies that are labeled with fluorescent dyes can also be used as labeling molecules. Antibodies are mostly directed against certain surface proteins, e.g., proteins of the CD classification (CD: cluster of differentiation). After labeling, the cells can then also be sorted according to these characteristics if necessary. By using different colored lasers and, above all, filters, the number of replaceable dyes and thus the information density can be increased. Antibodies are the most frequently used molecules to mark surface proteins on cells. Labeling molecules are understood to mean antibodies that are labeled with at least one optical marker and/or other molecules that are suitable for specifically binding to a target structure located on the surface of a cell contained in the analysis medium, such as a cancer cell, for example to label the cell optically.

Although the flow cytometer enables both the measurement of the number of labeled cells and the acquisition of a measured value for the fluorescence signal emitted by the individual cells in the case of fluorescence-labeled cells, a quantitative evaluation of the fluorescence signal is difficult because the flow cytometers used for the measurements are different, and therefore comparability of the flow cytometry measured values from measurements made with different flow cytometers is not guaranteed. In particular, the flow cytometry measured values are influenced by the intensity of the laser radiation used to illuminate the measurement region, by tolerances of optical filters, the structure of the flow measuring cell (fluidics), the reagents, detectors used and the variability of the samples containing the labeling molecules.

Although it is possible to quantify the fluorescence measurement values of the cells by comparing the first flow cytometry measured value with a threshold value, this is usually set arbitrarily. An exact quantification of the binding events per cell and thus a universal platform-independent comparability of the fluorescence measurement values has not been possible up to now.

Also already known is the calibration of flow cytometry measuring devices with the aid of fluorescent microparticles which are arranged in a fluid that is passed through the measurement region of the energy beam or the electric field. However, it turned out that this calibration is not sufficient to enable an exact comparison of the fluorescence readings measured with different flow cytometers.

### SUMMARY OF THE INVENTION

The object of the invention is therefore to provide a flow cytometry measuring method of the type mentioned at the outset which makes it possible to quantitatively detect the target structure on the surface of the cells with great precision. In addition, the quantitative measured values obtained by fluorescence measurement, if measured with different flow cytometers, should be precisely comparable with one another (referencing/normalizing). Another object of the invention is to provide a kit for carrying out the method.

This object is achieved by the use of a flow cytometry measurement method as described herein. According to the invention, a flow cytometry measuring method is provided in which an analysis medium is provided which has a fluid and biological cells to be classified contained therein, wherein at least one labeling molecule is provided and brought into contact with the analysis medium in such a way that the labeling molecule can specifically bind to at least one target structure located on the surface of the cells if the cell has this target structure, wherein a fluid flow of the analysis medium is generated in which the cells individually come into a measurement region of an energy beam and/or an electric field, wherein a first and a second flow cytometry measured value are captured respectively for a first and a second physical parameter measurement value for the individual cells located within the measurement region, wherein at least the first parameter is a fluorescent radiation which is emitted by the at least one labeling molecule when excited with the energy beam or the electric field, wherein the cells are classified on the basis of these flow cytometry measured values, wherein at least one first and at least one second calibrator are provided, each of which has a solid particle made of a water-insoluble, inorganic and/or polymeric material, wherein the solid particles of the at least one first and the at least one second calibrator match in terms of the shape, size, and material thereof, wherein the at least one first calibrator has at least one target structure on the surface thereof that matches the at least one target structure of the cells and is immobilized on the solid particle of the first calibrator, wherein the at least one second calibrator doesn't have this target structure, wherein the at least one first and at least one second calibrator being is mixed with the analysis medium before the flow cytometry measured values are captured in such a way that at least one labeling molecule located in the analysis medium binds to the at least one target structure of the first calibrator, wherein the calibrators in the fluid flow are introduced into the measurement region individually one after the other, wherein the first and second flow cytometry measured values corresponding to the at least one first and the at least one second calibrator are captured as for the cells, wherein the parameter assigned to the second flow cytometry measured value for the cells and the second flow cytometry measured value for the calibrators are selected such that the calibrators are distinguished based the second measured values from the cells, and wherein a normalized first flow cytometry measured value is formed for the at least one cell from the at least one first flow cytometry measured value for the at least one first calibrator, from the at least one first flow cytometry measured value for the at least one second calibrator, and from the first flow cytometry measured value from the at least one cell.

The invention is based on the knowledge that the tolerances of the reagents used to detect the binding of the labeling molecules to the target structure of the cells, the variability of the labeling molecules and the composition of the analysis medium have a significant influence on the flow cytometry measured values of the cells to be classified. These tolerances are determined in particular by the age of the labeling molecules, the manner in which and under which conditions the labeling molecules were stored before the flow cytometry measurement method was carried out, and by substances contained in the analysis medium in addition to the cells, such as inhibitors, which inhibit the binding and make it difficult for the labeling molecules to attach to the target structure, or to bind to the target structure instead of the labeling molecules. In addition, environmental conditions, such as temperature, can also affect the flow cytometry readings of the cells.

In the method according to the invention, these influences are compensated for in that standardized first and second calibrators are provided, that corresponding flow cytometry measured values are captured for these calibrators as for the cells, and that the flow cytometry measured values captured for the cells can be normalized with the aid of the flow cytometry measured values for the calibrators. This enables an exact quantification of the binding events occurring between the individual cells and the labeling molecules or the antibodies thereof per cell. The normalized flow cytometry measured values can also be compared exactly with one another in the case of flow cytometry measurements that were carried out with different flow cytometers and/or in which reagents or labeling molecules were used that differ from one another in terms of the properties thereof. This makes it possible, in particular, to identify cell sub-populations that belong together by means of cluster analysis. Since the calibrators are not living cells but have solid particles as a carrier for the at least one target structure, the calibrators have long-term stability, i.e., they can be stored for a longer period of time without significant changes in the properties thereof relevant to the detection of the target structure.

The parameter space of each analysis depends on the number of maximum binding events per cell and the measurement parameters. In other words, when determining three parameters, for example, these result in a three-dimensional space, the axes of which, however, are not necessarily at right angles to one another. The type of label and label density (number of fluorophores per antibody) and the binding strength and specificity of the antibodies (or labeling molecules) used also play a role.

In the method according to the invention, normalized flow cytometry measured values are provided for the cells, which are independent of the physical properties of the flow cytometer used to carry out the method, independent of tolerances of the reagents and labeling molecules used, and independent of substances and active ingredients which can be contained in the analysis medium in addition to the cells.

In an advantageous embodiment of the invention, to form the normalized first flow cytometry measured value, the difference between the first flow cytometry measured value for the first calibrator and the first flow cytometry measured value for the second calibrator is compared with the difference between the first flow cytometry measured value for the cell and the first the flow cytometry reading from the second calibrator. The normalized flow cytometry measured value for the cells is preferably 100% if the first flow cytometry measured value for the cells agrees with the first flow cytometry measured value for the first calibrator. If the first flow cytometry reading from the cells matches the first flow cytometry reading from the second calibrator, the normalized flow cytometry reading from the cells is zero. The first calibrators are preferably designed such that the first flow cytometry measured value for at least one first calibrator corresponds to the expected maximum value for the first flow cytometry measured value for the cells or is slightly higher, in particular a maximum of 3%, a maximum of 5%, a maximum of 10%, or a maximum of 25% above the expected first flow cytometry reading from the first calibrator. The corresponding expectation value can be determined experimentally.

In a preferred embodiment of the invention, multiple identical first calibrators and multiple identical second calibrators are provided and mixed with the analysis medium before the individual flow cytometry measured values are captured, wherein an averaged first flow cytometry from the first flow cytometry measured values is formed from the first calibrators measurement value for the first calibrators, and an averaged first flow cytometry measured value for the second calibrators is formed from the first flow cytometry measured values of the second calibrators, and
wherein, to form a normalized first measured value for a cell, the difference between the averaged first flow cytometry measured value for the first calibrators and the averaged first flow cytometry measured value for the second calibrators is compared with the difference between the first flow cytometry measured value for the cell and the averaged first flow cytometry measured value for the second calibrators,

or
wherein first flow cytometry measured values are captured for the individual cells to form a normalized first measured value for a cell population comprising multiple cells, wherein an averaged first flow cytometry measured value for the cell population is formed from these flow cytometry measured values, and wherein for the formation of the normalized first measurement value for multiple cells
   a) the difference between the averaged first flow cytometry measured value for the first calibrators and the averaged first flow cytometry measured value for the second calibrators is related to the difference between the first averaged flow cytometry measured value for the cell population and the averaged first flow cytometry measured value for the second calibrators, or
   b) the quotient is formed between the averaged first flow cytometry measured value and the averaged first flow cytometry measured value for a calibrator for a signal with a predetermined signal strength, in particular for a 100% signal.

The mean value can be determined using statistical methods known per se and in particular being the arithmetic mean value. If necessary, the normalized first measured value can also be scaled, wherein the scaling factor can correspond to the quotient of the averaged first flow cytometry measured value for the first calibrators and the number 100, for example.

In a further development of the invention, a first calibration factor is provided for the at least one first calibrator which, with respect to the measurement signal for the first parameter, corresponds to the ratio between the measurement signal strength of the first calibrator and the measurement signal strength of a first reference calibrator having the target structure physical parameters of the first calibrator, a first measurement signal is detected and the first flow cytometry measured value for the first calibrator is formed from the first measurement signal and the first calibration factor, wherein a second calibration factor is provided for the second calibrator which is related to the measurement signal for the first parameter and corresponds to the ratio between the measurement signal strength of the second calibrator and the measurement signal strength of a second reference calibrator that does not have the target structure, and wherein a second measurement signal is captured for the first physical parameter of the second calibrator and the first flow cytometry measured value for the second calibrator is formed from the second measurement signal and the second calibration factor.

The calibration factors make it possible to compare even more precisely flow cytometry measured values that were carried out with different batches of the first calibrator and/or the second calibrator differing from one another. The calibration factors are preferably measured experimentally under precisely defined conditions.

In this case, an expectation value for the first flow cytometry measured value is initially determined for the first and second reference calibrators that are produced in a first batch. Instead of the analysis medium, a buffer with defined, constant properties is used in which the first and second reference calibrators are arranged. The buffer does not contain biological cells. In this buffer, the corresponding first flow cytometry measured values are measured in each case for a plurality of identical or essentially identical first or second reference calibrators. From the reference flow cytometry measured values obtained in this way, an expectation value is determined for each of the first and second reference calibrators using known methods of statistics.

In a further step, expectation values are determined in a corresponding manner for the first and second calibrators of a second batch.

The first calibration factor is then determined by forming the quotient from the expectation value for the first flow cytometry measured values of the first calibrators of the second batch and the expectation value for the first flow cytometry measured values of the first reference calibrators. The second calibration factor is determined in a corresponding manner by forming the quotient from the expectation value for the first flow cytometry measured values of the second calibrators of the second batch and the expectation value for the first flow cytometry measured values of the second reference calibrators.

In an advantageous embodiment of the invention, at least two types of calibrators are provided by the first calibrators and mixed with the analysis medium, wherein the calibrators of the different types of calibrators differ from one another in particular with regard to the surface coverage density and/or the arrangement of their at least one target structure on the surface of the solid particle in such a way that the first flow cytometry measured values of a first calibrator type have a signal strength that is greater than the signal strength of the first flow cytometry measured values of the second calibrators and is less than the signal strength of the first flow cytometry measured values of a second calibrator type. As a result, the influence of a non-linear signal curve in the capture of the flow cytometry measured values can be compensated for.

It is advantageous if the fluid contains at least two different populations of cells which differ from one another in such a way that the first flow cytometry measured values of the cells of a first population lie within a first signal strength range and the first flow cytometry measured values of the cells of a second population lie within a second signal strength range located outside the first signal strength range, and if the signal strength of the first flow cytometry measured values of the first calibrator type is selected such that it lies between the first and second signal strength ranges. The calibrator of the first type of calibrator generates a signal strength that allows the reference ranges of two subpopulations to be delimited from one another. That is, cells with a first flow cytometry measured value that is smaller than the first flow cytometry measured value for the first calibrator of the first calibrator type belong to a first population and cells with a higher flow cytometry measured value belong to a second population.

In a preferred embodiment of the invention, the largest dimension of the solid particles is less than 20 pm, in particular less than 15 pm, and preferably less than 10 pm and/or the smallest dimension of the solid particles is greater than 4 pm, in particular greater than 5 pm, and is preferably greater than 6 pm. Suitable solid particles are, for example, microspheres with a diameter of 6-10 pm. The solid particles are preferably of a similar size to the cells in the analysis medium.

The solid particles are preferably synthetic microparticles. The solid particles can consist of a polystyrene, melamine, latex, and/or silicate, or consist of one of these materials.

The labeling molecules can be molecules that bind to target antigens on the surface of cells or calibrators and can then be used to detect these target antigens. Such molecules are, for example, antibodies and the fragments thereof, lectins, other binding proteins (e.g., protein A).

The target antigens on the first calibrators are preferably covalently immobilized. Processes for producing such a covalent bond are known from the prior art and include, for example, bonding to amines (via cross-linkers), thiols, epoxides, aldehydes, maleimides and other groups. Suitable processes and the chemical conversion thereof are described in the specialist literature (Bioconjugate Techniques, Third Edition, 3rd edition by Hermanson, Greg T. (2013)). However, non-covalent methods can also be used, such as, for example, His-Tag, biotin-streptavidin, protein G, protein A, pre-immobilized antibodies.

Target antigens or target structures on the first calibrators can be, for example, proteins, peptides, receptors, allergens, glycosylated proteins, liposaccharides, oligo- and polysaccharides, nucleic acids, and the fragments and derivatives of the aforementioned substances.

In a preferred embodiment of the invention, the at least one second flow cytometry measured value comprises a forward scatter measured value for a forward scatter of the energy beam occurring in the measurement region and/or a sideward scatter measured value for a sideward scatter of the energy beam occurring in the measurement region. These measured values can be captured in a simple manner using suitable sensors. The forward scatter measurement is a measure of the size and the sideward scatter measurement is a measure of the granularity of the cells or the calibrators. The cells can be distinguished from the calibrators on the basis of these measured variables. In this way, the measured flow cytometry measured values can be clearly assigned to the cells and the calibrators on the basis of the forward and sideward scatter measured values.

However, it is also possible that the at least one second flow cytometry measured value is a fluorescence measurement for a fluorescence radiation emitted by the labeling molecules, which differs from the fluorescence radiation of the first flow cytometry measured value. The wavelengths of the fluorescence radiation of the first and second flow cytometry measured values are preferably different.

It is advantageous if at least one first flow cytometry measured value is captured for multiple measurement channels for the cells as well as for the first and second calibrators, if at least two and in particular at least one number of different labeling molecules corresponding to the number of measurement channels is provided and is brought into contact with the analysis medium, and if the at least one first calibrator has at least one number of target structures corresponding to the number of measurement channels, which each specifically bind to one of the different labeling molecules when they come into contact with the labeling molecule in question.

However, it is also conceivable that at least one first flow cytometry measured value is captured for multiple measuring channels both for the cells and for the first and second calibrators, that at least one number of different labeling molecules corresponding to the number of measuring channels is provided and is brought into contact with the analysis medium that at least two types of calibrators are provided by first calibrators, that at least one first calibrator of a first type of calibrator has at least one first target structure on the surface thereof which corresponds to at least one first target structure of the cells and is immobilized on the solid particle of this calibrator, that at least one first calibrator of a second type of calibrator has on the surface thereof at least one second target structure which corresponds to at least one second target structure of the cells and is immobilized on the solid particle of this calibrator, and that the at least one first calibrator of the first calibrator type has the second target structure on the surface thereof and the at least one first calibrator of the second calibrator type does not have the first target structure on the surface thereof. In this case, at least a number of different first calibrators corresponding to the number of measurement channels can be provided and mixed with the analysis medium, and these first calibrators can have different target structures, each of which is binding-specific for one of the different labeling molecules.

The use of multiple measuring parameters and the calibrators thereof is only limited by the number of measuring channels of the measuring device and the available fluorophores. Six to eight parameters can be captured routinely. For the analysis of AML cells, 30 relevant parameters are already captured today, but they must be captured in multiple measurement runs. With modern devices that also allow measurements in the near infrared range, significantly more than eight parameters can be captured. If subclasses of cells are to be captured in such a parameter space, the resolution of the measurement is important. The number of measurements that can be captured per measurement channel can be increased by using calibrators of a further particle class, the solid particles of which differ in size from the solid particles of calibrators of a first particle class.

Because the first calibrators (relative signal strength 100%) allow the expected maximum signal to be captured in a device-specific manner, the resolution can be increased (e.g., by adapting the laser power or the amplifier power of the detector, so that the maximum resolution can be obtained from the measurement). With a signal captured and processed in this way, cell populations can now be captured with the help of mathematical methods (e.g., cluster analysis—a standard mathematical method, which is described at https://de.wikipedia.org/wiki/Clusteranalyse). Thus, very variable diseases such as leukemia can be better typed and thus also better treated, since appropriate markers for the response to therapies can be captured and evaluated. In the special case of lymphoma and leukemia, a number of markers are known which have a strong influence on the prognosis of the disease. Current studies show that in addition to known markers such as CD38, the expression of CD49d has a very serious impact on the course of chronic lymphocytic leukemia (CLL) (DOI: 10.1111/j.1365-2141.2011,08725.x).

In a preferred embodiment of the invention, the target structure has at least one antigen on the first calibrators, which preferably contains at least one protein and/or at least one peptide and/or at least one receptor and/or at least one allergen and/or at least one glycosylated protein and/or at least one liposaccharide and/or at least one oligosaccharide and/or at least one polysaccharide and/or at least one nucleic acid and/or at least one fragment and/or derivative of the aforementioned substances.

In an advantageous embodiment of the invention, the target structure of the at least one first calibrator is immobilized on the solid particle of the first calibrator via at least one activated carboxy groups and/or at least one activated NH2 group. This enables stable coupling and immobilization of the target structure, such as an antigen, to the solid particle. Corresponding microparticles are commercially available.

It is advantageous if at least one target structure immobilized on the solid particle of the first calibrator matches one of the target structures which is typical for cells of at least one type of blood cancer. The method according to the invention can then be used to classify leukemia cells. In particular, the rapidly growing type M cells can be distinguished from the less aggressive type D cells.

In a preferred embodiment of the invention, the at least one target structure immobilized on the solid particle of the first calibrator matches one of the target structures that occurs within the B-cell receptor (BCR) on the surface of chronic lymphatic leukemia cells (CLL cells). These leukemia cells are particularly aggressive. Due to the high affinity and specificity thereof, this target structure is well suited for flow cytometry diagnostics (FACS).

The calibrators are added to the analysis medium, e.g., blood, during the measurement. Suitable concentrations of the calibrators are preferably 1×10⁵ per 1×10⁶ cells. During the measurement, the calibrators are identified using the parameters forward scatter (size) and sideward scatter (granularity) and thus separated from the target cells.

With regard to the kit, the above-mentioned object is achieved with the features as described herein. According to the invention, the kit comprises
at least one first calibrator which has a first solid particle made of a water-insoluble, inorganic, and/or polymeric material, wherein the first calibrator has at least one target structure immobilized on the first solid particle on the surface thereof,
at least one second calibrator which has a second solid particle that corresponds to the first solid particle in terms of shape, size and material, the second calibrator not having the target structure on the surface thereof, and
at least one labeling molecule that binds specifically to the target structure.

With such a kit, target structures which are arranged on the surface of biological cells to be classified contained in a fluid can be captured quantitatively with great precision in flow cytometry measuring methods. The corresponding measured values can be compared precisely with one another—even if they were measured with different flow cytometers. The at least one target structure is preferably an antigen. If required, the kit can also contain a buffer.

In a preferred embodiment of the kit, multiple different target structures, which are immobilized on the first solid particle, are arranged on the surface of the at least one first calibrator. The kit has at least one binding-specific labeling molecule for each of these target structures. With such a kit, parallel measurements can be carried out. The parallel loading of the calibrators with target structures is only limited by the coverage density of the solid particles and thus by the maximum signal that can be achieved.

However, it is also conceivable that the kit for performing parallel measurements has multiple different first calibrators that have different target structures on the surface thereof. Each first calibrator can only have a single target structure. It is also possible for at least one first calibrator to have multiple regions with target structures which are binding-specific for the same labeling molecules.

The labeling molecules are preferably provided with fluorescent dyes. Such dyes are normally covalently bound to the labeling molecules. A large number of such dyes are commercially available, such as, for example, Cy3, Cy5, Cy7, FITC, rhodamine, phycoerythrin, lyssamine; a list of various dyes can be found at: https://en.wikipedia.org/wiki/Fluorophore.

In a preferred embodiment of the invention, the kit comprises at least one data carrier on which a first calibration factor for the at least one first calibrator and/or a second calibration factor for the at least one second calibrator is stored. Such a kit can be used to compensate for calibrator tolerances. This is particularly advantageous if the measurement results of flow cytometry measurement methods which were carried out with different batches of first or second calibrators are to be compared with one another. The data carrier can be a machine-readable data carrier which, for example, can have a carrier that is provided with a barcode or a QR code. However, it is also possible for the calibration factors to be printed in the form of numbers on a document belonging to the kit, such as an instruction leaflet or packaging belonging to the kit.

It is advantageous if the kit has at least two types of calibrators from the first calibrators, and if the calibrators of the different types of calibrators differ from one another, in particular with regard to the surface coverage density and/or the arrangement of the at least one target structure thereof on the surface of the solid particle, differing from one another in such a way that they generate measurement signals having different signal strengths if the target structures thereof are marked with the labeling molecule. With such a kit, a regression can be carried out for flow cytometry measurements in which the fluorescence signal has a non-linear course.

### BRIEF DESCRIPTION OF THE DRAWINGS

Further details, features and advantages of the present invention emerge from the following description of exemplary embodiments with reference to the drawing. In the figures:

FIG. 1 shows a graphic representation of measured values captured with the aid of a flow cytometer for a population of second calibrators (zero calibrator), wherein the measured intensity PE is plotted on the abscissa and the number n of binding events is plotted on the abscissa,

FIG. 2 shows a representation similar to FIG. 1, but wherein the measured values for a population of first calibrators of a first type of calibrator are shown,

FIG. 3 shows a representation similar to FIG. 1, but wherein the measured values for a population of first calibrators of a second type of calibrator are shown,

FIG. 4 shows a standard curve in which the amount of protein CD23 per 2.1·10⁶ solid particles is plotted on the abscissa and the MFI value (mean fluorescence measured value) is plotted on the ordinate,

FIG. 5 shows a graphic representation of the measurement signals measured for the parameters size and granularity, with the forward scatter (FSC) assigned to the parameter size on the abscissa and the sideward scatter (SSC) assigned to the parameter granularity of a fluorescence radiation on cells and calibrator particles on the ordinate,

FIG. 6 shows a graphic representation of the intensity values for IgM and CD19 measured for cells and calibrator particles, the intensity for IgM being plotted on the abscissa and the intensity for CD19 being plotted on the ordinate,

FIG. 7 shows a graphic representation of the intensity values for IgD and CD19 measured by cells and calibrator particles, the intensity for IgD being plotted on the abscissa and the intensity for CD19 being plotted on the ordinate,

FIG. 8 shows a graphic representation of the intensity values for IgD and IgM measured for cells and calibrator particles, wherein the intensity for IgD is plotted on the abscissa and the intensity for IgM is plotted on the ordinate,

FIG. 9 shows a three-dimensional representation of the measuring space according to FIGS. 6 to 8, and

FIG. 10 shows a graphical representation of a cluster analysis for the measurement space depicted in FIGS. 6 to 9.

### DESCRIPTION OF THE INVENTION

In a first exemplary embodiment of the invention, disease-associated antigens are provided as target structures. IgM (immunoglobulin M) is provided as a first target structure and IgD (immunoglobulin D) is provided as a second target structure.

In addition, a plurality of solid particles are provided, namely microspheres made of polystyrene with a diameter of 6 pm. The solid particles match with regard to their shape, their size and their material.

The solid particles are coated on the surface thereof with binding carboxyl groups (—COOH). Instead of or in addition to the carboxyl groups, NH₂ groups can be provided on the surface of the solid particles. The first and second target structures can be bound to the surface of the solid particles via the carboxyl groups and/or the NH₂ groups.

Furthermore, first and second labeling molecules are provided. The first labeling molecules are antigens that are binding-specific for the first target structure IgM. The second labeling molecules are antigens that are binding-specific for the second target structure IgD. The labeling molecules each have at least one optical marker which emits fluorescence radiation when excited with suitable excitation radiation.

1.1 Coupling of Disease-Associated Antigens to the Surface of the Solid Particles

Thirteen solid particle populations are provided, each of which comprises the same number of solid particles which have carboxyl groups on the surfaces thereof.

The first target structures are coupled to the surface of the solid particles from six solid particle populations and the second target structures are coupled to the surface of the solid particles from six further solid particle populations. For this purpose, there is added to the solid particles
of a first solid particle population, 2.00 μg IgM,
of a second solid particle population, 1.00 μg IgM,
of a third solid particle population, 0.40 μg IgM,
of a fourth solid particle population, 0.20 μg IgM,
of a fifth solid particle population, 0.08 μg IgM,
of a sixth solid particle population, 0.04 μg IgM,
of a seventh solid particle population, 2.00 μg IgD,
of an eighth solid particle population, 1.00 μg IgD,
of a ninth solid particle population, 0.40 μg IgD,
of a tenth solid particle population, 0.20 μg IgD,
of an eleventh solid particle population, 0.08 μg IgD and
of a twelfth solid particle population, 0.04 μg IgD, in each case added to the solid particles of the relevant solid particle population for every 1.2-10⁶ solid particles and incubated in a binding buffer (50 mM MES, pH 5.2; 0.05% proclin) for 60 minutes with constant agitation. The coupling takes place in each case according to a standard protocol by means of activation by EDAC (carbodiimide). The solid particles are washed and activated in EDAC solution (20 mg/mL).

After centrifugation of the individual solid particle populations at 800 g, the supernatant is removed in each case and set aside for a control measurement of the remaining antigen concentration. The solid particles are taken up in a washing buffer and resuspended. After another centrifugation, the supernatant is discarded and the particles are taken up in a washing and storage buffer (10mM Tris, pH 8.0; 0.05% BSA; 0.05% Proclin) to saturate unoccupied reactive groups.

Neither first nor second target structures are coupled to the surface of the solid particles of a thirteenth solid particle population. Instead, the reactive COOH groups are brought into contact with a blocking protein in such a way that the blocking protein can bind nonspecifically to the COOH groups. Bovine serum albumin or hydrolyzed casein, for example, can be used as blocking protein.

1.2 Determination of the Concentrations of IgM and IgD to be used for Coupling.

Each of the thirteen solid particle populations is measured using a flow cytometer (FACS). For this purpose, the solid particles of the first to sixth solid particle populations and the first labeling molecules (approx. 0.5 μg per measurement) are suspended in a fluid and incubated in the dark for 10 minutes. The samples are centrifuged and the supernatant discarded. It is washed once with 2 mL of PBS buffer (phosphate buffered saline) and then centrifuged again. After the supernatant has been discarded, the solid particles are taken up in 100-200 μL PBS. The suspension obtained in this way is passed through a nozzle opening of a flow cytometer that delimits a measurement region in such a way that the solid particles contained in the PBS individually reach a measurement region of a laser beam. The first labeling molecules specifically bound to the first target structure are excited by the laser beam to emit fluorescent radiation. Fluorescence measurement values are captured therefrom with the aid of an optical detector. A plurality of fluorescence measurement values (intensity measurement values) are measured for each solid particle population. A mean value (MFI value, or median fluorescence intensity) is formed from the fluorescence measured values captured for the individual solid particles for each solid particle population.

In a corresponding manner, the solid particles of the seventh to twelfth solid particle population are measured with the aid of the flow cytometer. Thus the second labeling molecules specifically bound to the second target structure are excited by the laser beam to emit fluorescent radiation.

Although human IgM (immunoglobolin M) is easily accessible from serum, it turned out that for the coupling to the solid particles and in particular for the stability after the coupling, monomeric human IgM produced recombinantly is more suitable. IgM in serum is usually present as a pentamer. It was found that the binding of IgM pentamers usually does not lead to complete binding. Some of the IgM molecules in the pentamers are not covalently bound to the particles. Uncontrolled degradation then leads to a loss of resolution in the flow cytometer during later use. Thus, for both IgM and IgD (IgD (immunoglobolin D) monomeric IgM and IgD produced recombinantly are used.

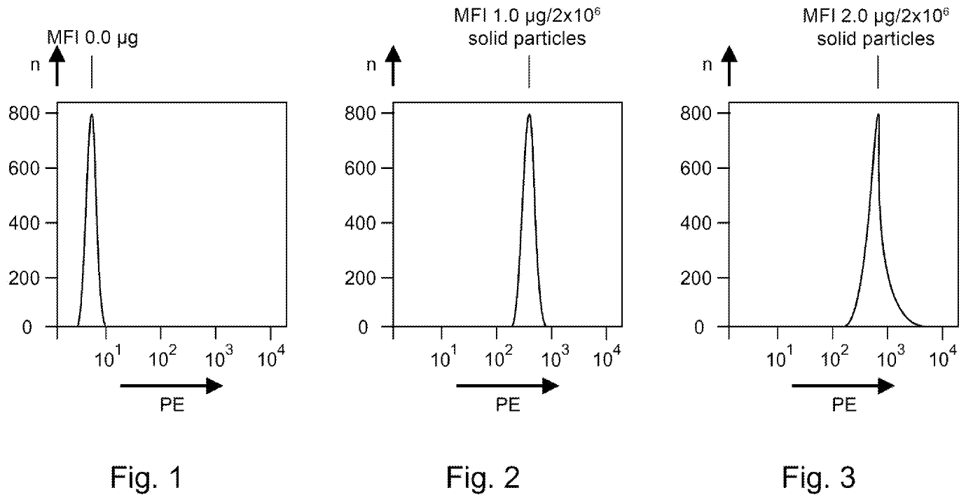

In FIGS. 1 to 3, the fluorescence measurement values and the mean value for the first, second, and seventh solid-state particle populations are shown graphically. The intensity PE of the fluorescent radiation measured with the aid of the flow cytometer for the solid particles of the solid particle population in question is plotted on the abscissa and the number n of binding events is plotted on the abscissa.

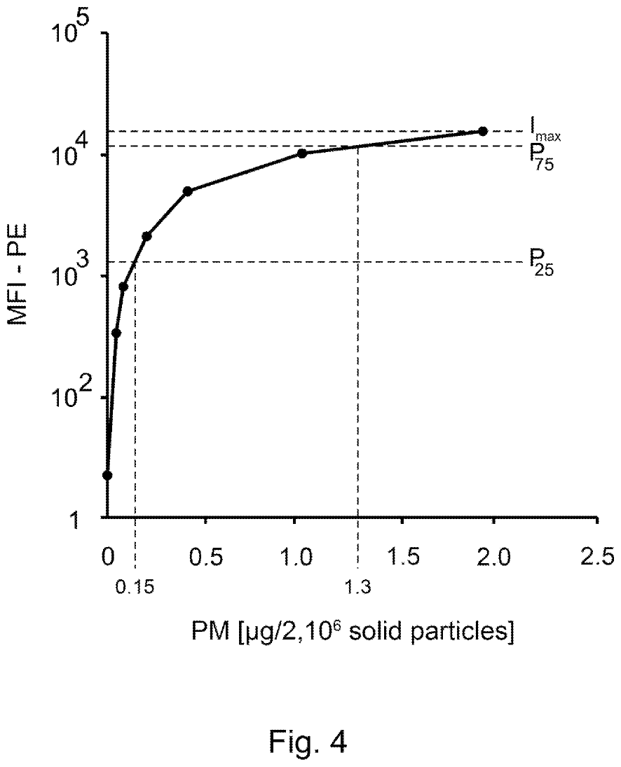

As can be seen in FIG. 4, the mean values for the individual fluorescences are plotted to form a coupling curve against the CD23 protein amount PM per 2.1·10⁶ solid particles in each case for the first. A corresponding coupling curve is created for the second target structure.

The quantity of the first or second target structure required for a desired intensity or signal strength of the fluorescence can be read from the relevant coupling curve. The maximum mean value of the intensity Iₘₐₓ for the respective target structure is determined and defined on the basis of the coupling curve. Then, a 75% value (P₇₅) and a 25% value (P₂₅) are defined as follows:

P₇₅=Iₘₐₓ·0.75

P₂₅=P₇₅/10

The following values result for the coupling in the first exemplary embodiment:

Iₘₐₓ=21,700

P₇₅=21700·0.75=16275

P₂₅=16275/10=1627

These values are set as a reference. The appropriate amount of protein can now be read from the coupling curve. For P₇₅ this is 1.3 μg per 2.1·10⁶ solid particles.

1.3 Preparation of Calibrators for Two Parameters Characteristic of CLL: IgM and IgD

The aim is to create multiple calibrator populations for each of the first and second target structures (IgM and IgD).

A first calibrator population contains a plurality of matching first calibrators of a first calibrator type. These calibrators each have a solid particle on the surface of which IgM is immobilized as the first target structure in such a way that the fluorescence signal of the individual first calibrators of the first calibrator type reaches a relative intensity of 25% in a flow cytometry analysis.

A second calibrator population contains a plurality of matching first calibrators of a second calibrator type. These calibrators each have the same solid particles as the first calibrators of the first type of calibrator. The same target structure is immobilized on the surface of the solid particles of the first calibrators of the second calibrator type as on the surface of the first calibrators of the first calibrator type. The surface coverage density and the arrangement of the target structure of the first calibrators of the second calibrator type is selected such that the fluorescence signal of the first calibrators of the second calibrator type reaches a relative intensity of 75% in the flow cytometry analysis.

A third calibrator population contains a plurality of matching first calibrators of a third calibrator type. These calibrators each have the same solid particles as the first calibrators of the first and second calibrator types. A second target structure specific for IgD binding is immobilized on the first calibrator of the third calibrator type in such a way that the fluorescence signal reaches a relative intensity of 25% in the flow cytometry analysis of the first calibrators of the fourth calibrator type.

A fourth calibrator population contains a plurality of matching first calibrators of a fourth calibrator type. These calibrators each have the same solid particles as the first calibrators of the first, second, and third types of calibrator. The same target structure is immobilized on the surface of the solid particles of the first calibrators of the fourth calibrator type as on the surface of the first calibrators of the third calibrator type. The surface coverage density and the arrangement of the target structure of the first calibrators of the fourth calibrator type is selected such that the fluorescence signal of the first calibrators of the fourth calibrator type reaches a relative intensity of 75% in the flow cytometry analysis.

In addition, a fifth calibrator population is created which contains a multiplicity of matching first calibrators of a fifth calibrator type. These calibrators each have the same solid particles as the first calibrators of the first, second, third, and fourth calibrator types. Two different target structures are immobilized on the surface of the solid particles of the first calibrators of the fifth calibrator type. One of these target structures is identical to the first target structure (binding-specific for IgM) and the other target structure is identical to the second target structure (binding-specific antigen for IgD). The surface density and the arrangement of the antigens IgM and IgD of the first calibrators of the fifth calibrator type is selected such that the fluorescence signal of the first calibrators of the fifth calibrator type in the flow cytometry analysis reaches a relative intensity of 75% for both IgM and IgD.

In addition, a negative population is created which has a plurality of matching second calibrators. These each consist of a solid particle on which neither the first (IgM) nor the second target structure (IgD) is immobilized. In the negative population, the carboxyl groups (—COOH) or NH₂ groups on the surface of the solid particles are non-specifically bound to a blocking protein. The solid particles of the second calibrators are identical to the solid particles of the first calibrators.

For the coupling of the antigens to the solid particles, the amount of solid particles required for each population to be created is transferred to a reaction vessel. The solid particles are centrifuged at 800 g for 5 minutes and the supernatant is discarded. The solid particles are then taken up in a coupling buffer. The coupling takes place according to the protocol described above, wherein an adequate amount of IgM is used for the first, second, and fifth calibrator population and an adequate amount of IgD is used for the third, fourth, and fifth calibrator population.

The calibrator populations provided in this way are listed below:

First Calibrators:
a) IgM calibrators: PD₂₅—IgM concentration Int₂₅
PD₇₅—IgM concentration Int₇₅
b) IgD calibrators: PD₂₅—IgD concentration Int₂₅
PD₇₅—IgD concentration Int₇₅
c) IgM and IgD calibrators: P_{md}—IgM and IgD concentration Int₇₅
Second calibrators: P₀—neither IgM nor IgD

The individual calibrators are measured according to the position thereof by means of FACS (fluorescence-activated cell sorting) using commercially available antibodies marked with a marker. The calibrators are combined into a kit for use in later diagnostics. In the later FACS measurement, for diagnostic purposes, the measurement space is defined, standardized, and normalized by the individual populations.

1.4 Use of the Calibrators to Analyze Blood Samples Using FACS

A Kit is Provided that Includes:
a) A first population of first calibrators of the first calibrator type, which comprises a multiplicity of the first PD₂₅ calibrators described in Section 1.3, on the surface of which IgM is immobilized as the first target structure.
b) A second population of first calibrators of the first calibrator type, which comprises a multiplicity of the first PD₇₅ calibrators described in Section 1.3, on the surface of which IgM is immobilized as the first target structure.
c) A first population of first calibrators of the second calibrator type, which comprises a multiplicity of the first PD₂₅ calibrators described in Section 1.3, on the surface of which IgD is immobilized as the first target structure.
d) A second population of first calibrators of the second calibrator type, which comprises a multiplicity of the first PD₇₅ calibrators described in Section 1.3, on the surface of which IgD is immobilized as the first target structure.
e) A population of second calibrators comprising a plurality of the second calibrators P₀ described in Section 1.3.
f) A population of first labeling molecules, each of which has a first antigen which binds specifically to the first target structure IgM. The first labeling molecules each have a first optical marker which is coupled to the first antigen and emits a first fluorescent radiation when excited by a suitable first excitation radiation.
g) A population of second labeling molecules, each of which has a second antigen which binds specifically to the second target structure IgD. The second labeling molecules each have a second optical marker which is coupled to the second antigen and emits a second fluorescence radiation when excited with a suitable second excitation radiation.
h) a phosphate buffered saline solution (PBS buffer).

In the case of a FACS-based analysis of blood cells, the number of cells to be classified (lymphoma cells) is determined in the blood analysis medium after the blood has been drawn and the required number of cells for each FACS analysis is placed in a reaction vessel (e.g., 1×10⁶ cells per measurement). The labeling molecules (approx. 0.5 pg per measurement) and the calibrators P₀, PD₂₅, PD₇₅ are then added and incubated for 10 minutes in the dark. After the incubation time, the volume is made up with 2 mL of lysis/fixing buffer and incubated again for 10 minutes. In this step, not only are the cells fixed, but the majority of the red blood cells are made to burst. These are not important for the measurement and might interfere therewith.

The samples are centrifuged and the supernatant discarded. It is washed once with 2 mL of PBS buffer (phosphate buffered saline) and then centrifuged again. After the supernatant has been discarded, the cells are taken up in 100-200 μL PBS.

The analysis medium obtained in this way is then analyzed in a FACS flow cytometer. In the FACS flow cytometer, the analysis medium is passed through a nozzle opening delimiting a measurement region in such a way that both the cells marked with the labeling molecule and the different calibrators each come individually into the measurement region of the laser beams.

With the aid of the FACS flow cytometer, one first and two second flow cytometry measured values are captured for each calibrator entering the measurement region and for each cell entering the measurement region. The first flow cytometry measured value is dependent on the fluorescence radiation, which is emitted by at least one labeling molecule bound to the relevant calibrator located in the measurement region or the relevant cell located in the measurement region due to the excitation by the laser beam. The two second flow cytometry measured values include a forward scatter measured value and a sideward scatter measured value for the scattered radiation generated when the laser beam strikes the relevant calibrator located in the measurement region or the relevant cell located in the measurement region.

The second flow cytometry measured values are compared with reference values. Depending on the result of the comparison, the different scattering properties of cells and calibrators can be used to determine whether the second flow cytometry measured values were caused by scattering of the laser beam on a cell or on a calibrator. The different calibrators can be distinguished on the basis of the signal level of the first flow cytometry measured values thereof. Accordingly, depending on the result of the comparison, the first flow cytometry measured value is assigned either to the cells or to a calibrator.

If it is a calibrator, the first flow cytometry measured value is compared with comparison intervals which are assigned to the various calibrator particle populations. Thus, the first measured flow cytometry value can be assigned to a calibrator of a specific calibrator particle population. The assignment can take place automatically, for example by means of suitable software.

For each individual target structure (IgM or IgD), the respective mean values of the first flow cytometry measured values (fluorescence intensities) are determined for the individual calibrators P₀, PD₂₅, and PD₇₅. These form the basis for the reference measuring room used for the measurement. The first flow cytometry reading for the calibrators P₀ is used to determine the background in the channel. In addition, the first flow cytometry measured value for the calibrators P₀ enables a statement to be made about the quality of the coupling of the antigens of the labeling molecules to the target structure assigned thereto (negative control).

In addition, mean values of the first flow cytometry measured values are determined for the cells for each individual target structure.

In the exemplary embodiment, the following mean values are determined for IgM for the first flow cytometry measured values:
First PD₂₅ calibrators of the first type of calibrator: 1600
First PD₇₅ calibrators of the first type of calibrator: 16000
Second calibrators P₀: 25
Cells for the first target structure: 5600

First, the background noise is removed from the measured values. The background noise is caused by nonspecific binding to the solid particles of the calibrators or to the cells of the antibodies of the labeling molecules used. For this purpose, the first flow cytometry measured values of the first calibrators P₀ of the first calibrator type are subtracted from the first flow cytometry measured values of the first calibrators PD₂₅ and PD₇₅ of the first calibrator type, the second calibrators P₀ and the cells. The following adjusted first flow cytometry measured values result for IgM:
First PD₂₅ calibrators of the first type of calibrator: 1600-25=1575
First PD₇₅ calibrators of the first type of calibrator: 16000-25=15975
Second calibrators P₀: 25-25=0
Cells for the first target structure: 5600-25=5575.

Reference units are defined for the calibrators PD₂₅ and PD₇₅. 100 reference units are assigned to the adjusted first flow cytometry measured value for the calibrator PD₇₅ of the first calibrator type. A reference unit thus corresponds to an adjusted first flow cytometry measured value of 15975/100=159.75. This number is also referred to as the first scaling factor in the following. The adjusted first flow cytometry measured value for the calibrator PD₂₅ of the first calibrator type corresponds to 1575/159.75=9.86 reference units. A first normalized measured value is formed from the adjusted first flow cytometry measured value for the first target structure of the cells and the first scaling factor: 5575/159.75=34.90 reference units.

In the exemplary embodiment, the following mean values are determined for IgD for the first flow cytometry measured values:
First calibrators PD₂₅ of the second calibrator type: 3103
First calibrators PD₇₅ of the second calibrator type: 31030
Second calibrators P₀: 144
Cells for the second target structure: 4450

The following adjusted first flow cytometry measured values result for IgD:
First calibrators PD₂₅ of the second calibrator type: 3103-144=2959
First calibrators PD₇₅ of the second calibrator type: 31030-144=30886
Second calibrators P₀: 144-144=0
Cells for the second target structure: 4450-144=4306.

Reference units are again defined for the calibrators PD₂₅ and PD₇₅. 100 reference units are assigned to the adjusted first flow cytometry measured value for the calibrator PD₇₅ of the second calibrator type. A reference unit thus corresponds to an adjusted first flow cytometry measured value of 30886/100=308.86. This number is also referred to below as the second scaling factor. The adjusted first flow cytometry measured value for the calibrator PD₂₅ of the second calibrator type corresponds to 2959/308.86=9.58 reference units. A second normalized measured value is formed from the adjusted first flow cytometry measured value for the second target structure of the cells and the second scaling factor: 4306/308.86=13.94 reference units.

By assigning the reference units, the adjusted first flow cytometry measured values of the cells are independent of the properties and settings of the flow cytometer used and can be compared with corresponding adjusted first flow cytometry measured values obtained with a flow cytometer that is set differently than the first mentioned flow cytometer or which has other properties than this.

In a later step, if necessary, the standardized measured values of the cells related to the reference units can be related to those of the coupling curve (FIG. 4). The coupling curve corresponds to a logistic function (Gauss-Lorentz distribution). With the help of a “best-fitting” curve, absolute values can be determined from the values specified in reference units, which correspond to the quantity of the relevant target structure located on the cells.

2.1 Preparation of Calibrators with Three CLL-Characteristic Parameters IgM, IgD, and CD19

In a second exemplary embodiment, the number and the combination of the individual populations are expanded compared to the first exemplary embodiment for the use of further parameters. As described for IgM and IgD, a standard curve for the effective intensity was first created for the human and recombinantly produced B-lymphocyte antigen CD19. The appropriate amounts in relation thereto were used for the coupling.

First Calibrators:
a) IgM calibrators: PD₂₅13 IgM concentration Int₂₅
PD₇₅—IgM concentration Int₇₅
b) IgD calibrators: PD₂₅—IgD concentration Int₂₅
PD₇₅—IgD concentration Int₇₅
c) CD19 calibrators: PD₂₅—CD19 concentration Int₂₅
PD₇₅—CD19 concentration Int₇₅
d) IgM and IgD calibrators: P_{md}—IgM and IgD concentration Int₇₅
e) CD19 and IgM calibrators: P_{CD19/m}—CD19 and IgM concentration
Int₇₅
f) CD19 and IgD calibrators: P_{CD19/d}—CD19 and IgD concentration Int₇₅

Second Calibrators: P₀—neither IgM nor IgD nor CD19

This method can be extended and varied to any number of parameters.

2.2 Schematic Representation of Immunoprofiling Using Calibrator Particles

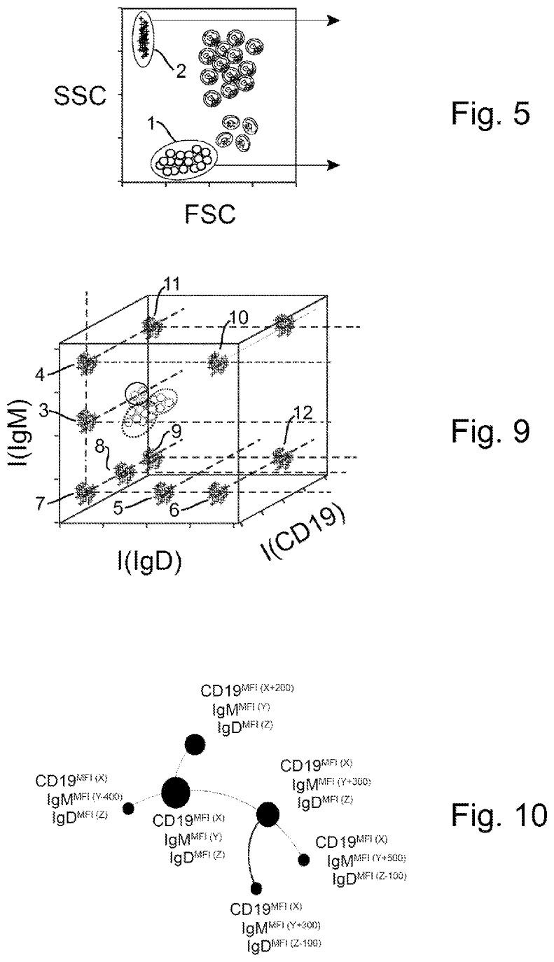

The cells to be classified and the calibrator particles are measured and analyzed simultaneously in the flow cytometer. As can be seen in FIG. 5, in a first step the measurement signals of the cells and the measurement signals of the calibrator particles are separated according to the size and granularity parameters. The size is represented by the forward scatter measurement values (FSC) of the energy beam and the granularity is represented by the sideward scatter measurement values (SSC) of the energy beam. In this phase, the cells to be analyzed and the calibrator particles are selected (gating). The measured values of the selected cells (cell populations) are delimited in FIG. 5 by a first oval 1 and the measured values of the selected calibrators (calibrator populations) by a second oval 2.

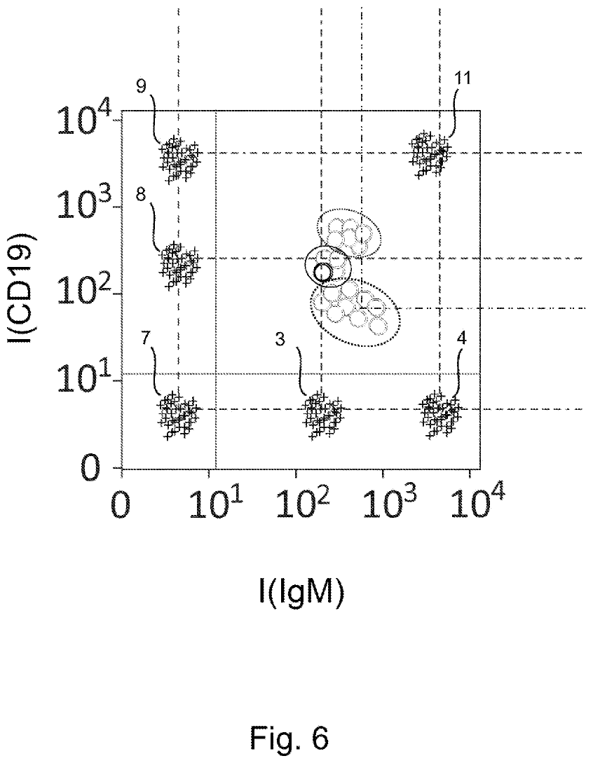

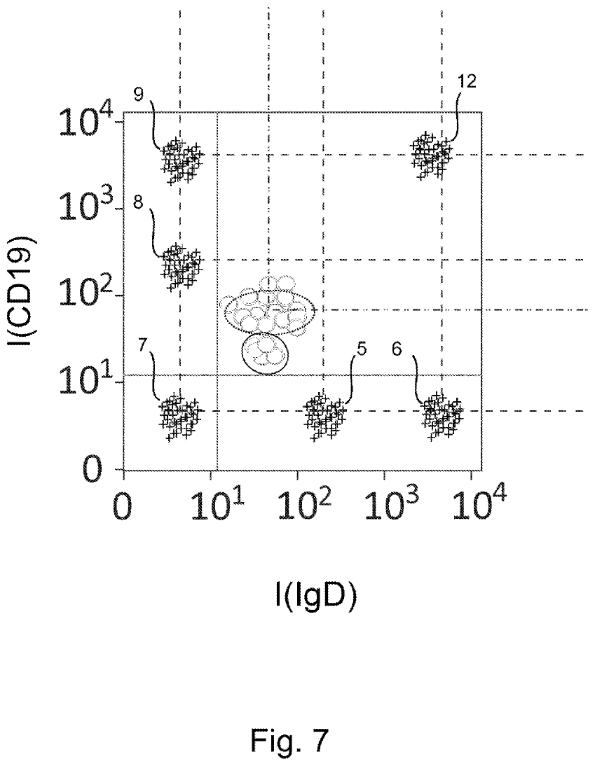

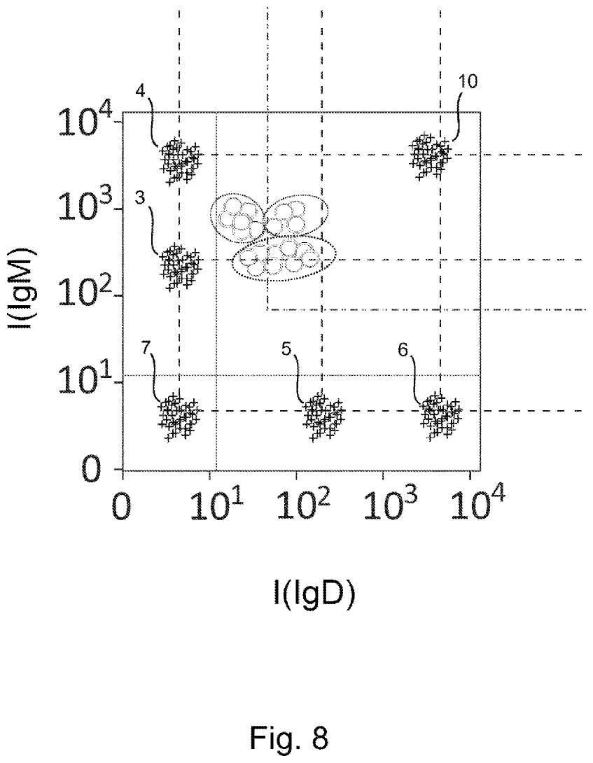

These populations can now be analyzed together for the various parameters (color channels). The calibrator particles form a reference grid for each channel. In FIGS. 6 to 9, the exemplary analysis of three parameters (IgM, IgD and CD19) is shown graphically, where
l(lgM) is the mean intensity measured for IgM,
l(lgD) is the mean intensity measured for IgD and
I(CD19) is the mean intensity measured for the antigen CD19

Of the fluorescence radiation. The measured values of the first population of first calibrators PD₂₅—IgM of the first calibrator type are denoted by the reference number 3, the measured values of the second population of first calibrators PD₇₅—IgM of the first calibrator type denoted by the reference number 4, the measured values of the first population of first calibrators PD₂₅—IgD of the second calibrator type denoted by reference number 5, the measured values of the second population of first calibrators PD₇₅—IgD of the second calibrator type denoted by reference number 6, and the measured values of the population of second calibrators P₀ are denoted by reference number 7. The measured values of the calibrators PD₂₅—CD19 are denoted by the reference number 8, the measured values of the calibrators PD₇₅—CD19 denoted by the reference number 9, the measured values of the calibrators P_{md}—IgM and IgD denoted by the reference number 10, the measured values of the calibrators P_{CD19/m}—CD19 and IgM denoted by the reference number 11, and the measured values of the calibrators P_{CD19/d}-—CD19 and IgD denoted by the reference number 12.

The calibrator particles serve multiple purposes:
1. Positive and negative controls for the staining properties of the detection antibodies used under identical conditions compared to the cells.
2. Definition and standardization of the measuring space, whereby
   i) an independence from certain devices and/or manufacturers is achieved,
   ii) a profile of the cells to be analyzed with regard to the parameters to be analyzed (in the example, IgM, IgD, CD19) can be created, and
   iii) the basis for an automated analysis is laid.

With regard to the multiparametric measuring space defined and standardized in this way (FIG. 9), the values of the cells are normalized and can be combined into groups or clusters on the basis of the profiles thereof. This clustering is the basis for creating an antigen profile of the cells to be analyzed.

The result of the cluster analysis is shown graphically in FIG. 10. Each circle represents a clustered subpopulation, the diameter of the circle being a measure of the number of measured values belonging to the relevant subpopulation in relation to the total number of measured values. The information relates to the mean fluorescence intensity (MFI) of a starting population, which is marked by the circle with the largest diameter. This starting population has MFI values X, Y, and Z for CD19, IgM, and IgD. The other subpopulations have a correspondingly altered MFI value. So if the value for the initial population were X=1000, then a cluster with X+200 would have an MFI of 1200 for the parameter in question.

1. A flow cytometry measuring method in which an analysis medium is provided which has a fluid and biological cells to be classified contained therein, at least one labeling molecule being provided and brought into contact with the analysis medium in such a way that the labeling molecule can specifically bind to at least one target structure located on the surface of the cells can bind if the cell has this target structure, a fluid flow of the analysis medium being generated in which the cells individually come into a measurement region of an energy beam and/or an electric field, a first and a second flow cytometry measured value being captured respectively for a first physical parameter and a second physical parameter for the individual cells located in the measurement region, at least the first parameter being a fluorescence radiation emitted by the at least one labeling molecule when excited with the energy beam or the electric field, and the cells are classified on the basis of the flow cytometry measured values, wherein at least one first and at least one second calibrator are provided, each of which has a solid particle made of a water-insoluble, inorganic and/or polymeric material, that the solid particles of the at least one first and the at least one second calibrator match in terms of the shape, size, and material thereof, that the at least one first calibrator has at least one target structure on the surface thereof, that matches the at least one target structure of the cells and is immobilized on the solid particle of the first calibrator, that the at least one second calibrator does not have this target structure, that the at least one first and the at least one second calibrator are mixed with the analysis medium such that before the flow cytometry measured values are captured in such a way that at least one labeling molecule located in the analysis medium binds to the at least one target structure of the first calibrator, that the calibrators in the fluid flow are introduced into the measurement region one after the other, that the first and second flow cytometry measured values corresponding to the at least one first and the at least one second calibrator are captured as for the cells, that the parameter assigned to the second flow cytometry measured value for the cells and the second flow cytometry measured value for the calibrators is selected, that the calibrators based on the second measurement values from the cells can be distinguished, and that from the at least one first flow cytometry measured value for the at least one first calibrator, the at least one first flow cytometry measured value for the at least one second calibrator, and the first flow cytometry measured value for at least one cell forms a normalized first flow cytometry measured value for the at least one cell.

2. The flow cytometry measuring method according to claim 1, wherein for the formation of the normalized first flow cytometry measured value, the difference between the first flow cytometry measured value for the first calibrator and the first flow cytometry measured value for the second calibrator is compared to the difference between the first flow cytometry measured value for the cell and the first flow cytometry reading of the second calibrator.

3. The flow cytometry measuring method according to claim 1, wherein multiple identical first calibrators and multiple identical second calibrators are provided and mixed with the analysis medium before the flow cytometry measured values are captured for the individual, that an averaged first flow cytometry measured value for the first calibrators is formed from the first flow cytometry measured values of the first calibrators and an averaged first flow cytometry measured value for the second calibrators is formed from the first flow cytometry measured values of the second calibrators, and that to form the normalized first measurement value for a cell, the difference between the averaged first flow cytometry measured value for the first calibrators and the averaged first flow cytometry measured value for the second calibrators is compared to the difference between the first flow cytometry measured value for the cell and the averaged first flow cytometry metric measured value for the second calibrators.

4. The flow cytometry measuring method according to claim 1, wherein a first calibration factor is provided for the at least one first calibrator, which with respect to the measurement signal for the first parameter corresponds to the ratio between the measurement signal strength of the first calibratorand the measurement signal strength of a first reference calibrator having a target structure so that a first measurement signal is captured for the first physical parameter of the first calibrator and the first flow cytometry measured value for the first calibrator is formed from the first measurement signal and the first calibration factor, that a second calibration factor is provided for the second calibrator, which with regard to the measurement signal for the first parameter, corresponds to the ratio between the measurement signal strength of the second calibrator and the measurement signal strength of a second calibrator that does not have the target structure, and that for the first physical parameters of the second calibrator, a second measurement signal is detected and the first flow cytometry measured value for the second calibrator is formed from the second measurement signal and the second calibration factor.

5. The flow cytometry measuring method according to claim 1, wherein at least two types of calibrators are provided by the first calibrators and are mixed with the analysis medium, so that the calibrators of the various types of calibrators differ from each other in particular with regard to the surface coverage density and/or the arrangement of the at least one target structure thereof on the surface of the solid particle in such a way that the first flow cytometry measured values of a first type of calibrator have a signal strength that is greater than the signal strength of the first flow cytometry measured values of the second calibrators and is smaller than the signal strength of the first flow cytometry measured values of a second calibrator type.

6. The flow cytometry measuring method according to claim 5, wherein the fluid contains at least two different populations of cells which differ from one another in such a way that the first flow cytometry measured values of the cells of a first population lie within in a first signal strength range and the first flow cytometry measured values of the cells of a second population lie within a second signal strength range located outside of the first signal strength range, and that the signal strength of the first flow cytometry measured values of the first calibrator type is selected such that it lies between the first and second signal strength range.

7. The flow cytometry measuring method according to claim 1, wherein the largest dimension of the solid particles is less than 20 μm, in particular less than 15 μm and preferably less than 10 μm and/or that the smallest dimension of the solid particles is greater than 4 μm, in particular larger than 5 μm and preferably larger than 6 μm.

8. The flow cytometry measuring method according to claim 1, wherein the material of the solid particles is polystyrene, melamine, latex, or a silicate.

9. The flow cytometry measuring method according to claim 1, wherein the at least one second flow cytometry measured value is a forward scatter measured value for a forward scatter of the energy beam occurring within the measurement region and/or a sideward scatter measured value for a sideward scatter of the energy beam occurring in the measurement region.

10. The flow cytometry measuring method according to claim 1, wherein for the cells as well as for the first and second calibrators in each case for multiple measuring channels, at least one first flow cytometry measured value is captured that at least one number of the number of measuring channels corresponding to the number of different labeling molecules is provided and brought into contact with the analysis medium, and that the at least one first calibrator has at least two and in particular at least one number of target structures corresponding to the number of measurement channels, which each specifically bind to one of the different labeling molecules when they come into contact with the relevant labeling molecule.

11. The flow cytometry measuring method according to claim 1, wherein at least one first flow cytometry measured value is captured for the cells as well as for the first and second calibrators in each case for multiple measuring channels, that at least one of the number of measuring channels corresponding to the number of different labeling molecules is provided and brought into contact with the analysis medium, that at least two types of calibrators are provided by first calibrators, that at least one first calibrator of a first type of calibrator has at least one first target structure on the surface thereof that corresponds to at least one first target structure of the cells and is immobilized on the solid particle of this calibrator such that at least a first calibrator of a second calibrator type has at least one second target structure on the surface thereof which corresponds to at least one second target structure of the cells and is immobilized on the solid particle of this calibrator, and that the at least one first calibrator of the first calibrator type has the second target structure on the surface thereof and the at least one first calibrator of the second calibrator type does not have the first target structure on the surface thereof.

12. The flow cytometry measuring method according to claim 1, wherein the target structure on the first calibrators has at least one antigen, which preferably contains at least one protein and/or at least one peptide and/or at least one receptor and/or at least one allergen and/or at least one glycosylated protein and/or at least one liposaccharide and/or at least one oligosaccharide and/or at least one polysaccharide and/or at least one nucleic acid and/or at least one fragments and/or derivatives of the aforementioned substances.

13. The flow cytometry measuring method according to claim 1, wherein the target structure of the at least one first calibrator is immobilized on the solid particle of the first calibrator via at least one activated carboxy group and/or at least one activated NH2 group.

14. A kit for performing the method of claim 1, comprᵢₛing
at least one first calibrator which has a first solid particle made of a water-insoluble, inorganic, and/or polymeric material, wherein the first calibrator has at least one target structure immobilized on the first solid particle on the surface thereof,
at least one second calibrator which has a second solid particle that corresponds to the first solid particle in terms of shape, size and material, the second calibrator not having the target structure on the surface thereof, and
at least one labeling molecule that binds specifically to the target structure.

15. The kit according to claim 14, wherein the kit comprises at least one data carrier on which a first calibration factor is stored for the at least one first calibrator and/or a second calibration factor is stored for the at least one second calibrator.

16. The kit according to claim 14, wherein the kit has at least two types of calibrators from the first calibrators, that the calibrators of the different types of calibrators differ from one another, in particular with regard to the surface coverage density and/or the arrangement of their at least one target structure on the surface of the solid particle, and that in a flow cytometry measurement they generate measurement signals with different signal strengths if the target structures thereof are labeled with the labeling molecule.
